(19) United States
(12) Patent Application Publication
(10) Pub. No.: US 2008/0085250 A1
(43) Pub. Date: Apr. 10, 2008

(54) Assortment Of Antiperspirants Having Varying Product Performance Characteristics

(75) Inventors: Timothy Alan Scavone, Loveland, OH (US); Theresa Louise Johnson, South Lebanon, OH (US); Michael Edward Thomas, Cincinnati, OH (US); Anne Marie Westbrook, Mason, OH (US)

Correspondence Address: THE PROCTER & GAMBLE COMPANY;INTELLECTUAL PROPERTY DIVISION - WEST BLDG., WINTON HILL BUSINESS CENTER - BOX 412, 6250 CENTER HILL AVENUE, CINCINNATI, OH, 45224, US

(73) Assignee: The Procter and Gamble Company, Cincinnati, OH (US)

(21) Appl. No.: 11/958,583

(22) Filed: Dec. 18, 2007

Related U.S. Application Data
(63) Continuation of application No. 11/818,019, filed on Jun. 13, 2007.
Application No. 11/818,019 is a continuation-in-part of application No. 11/598,405, filed on Nov. 13, 2006.
Application No. 11/598,405 is a continuation-in-part of application No. 11/451,702, filed on Jun. 13, 2006.
Application No. 11/451,702 is a continuation-in-part of application No. 11/150,980, filed on Jun. 13, 2005.

(51) Int. Cl. A61K 8/00; A61Q 15/00

(52) U.S. Cl. 424/65

(57) ABSTRACT

An array of antiperspirant products, comprising a first antiperspirant product that is marketed to male consumers and marketed as clinical strength or prescription strength; and a second antiperspirant product that is marketed to female consumers and marketed as clinical strength or prescription strength.

### CROSS REFERENCE TO RELATED APPLICATION

This application is a continuation of U.S. application Ser. No. 11/818,019, filed Jun. 13, 2007, which is a continuation-in-part of U.S. application Ser. No. 11/598,405, filed Nov. 13, 2006, which is a continuation-in-part of U.S. application Ser. No. 11/451,702, filed on Jun. 13, 2006, which is a continuation-in-part of U.S. application Ser. No. 11/150,980, filed Jun. 13, 2005.

### FIELD OF THE INVENTION

The present invention is related to a line of consumer products, particularly, antiperspirants having varying performance characteristics comprising a sequence of designs and packaging to aid a consumer in identifying the product and/or composition so desired.

### BACKGROUND OF THE INVENTION

The number of different antiperspirant products being offered for sale at any one location can be overwhelming to prospective buyers. Furthermore, it is far from clear the level of wetness and/or odor protection one may get by choosing one product over another product. Manufacturers may employ a variety of different marketing tactics utilizing product performance terms or other indicia in an attempt to make their particular product stand out, or to suggest to consumers that the product is “better” compared to the other products on the shelf. However, the marketing terms and/or indicia may not translate into actual differences in wetness or malodor protection. Thus, if consumers truly desire to determine which products actually perform significantly better regarding wetness and/or malodor protection than alternative products, they will likely need to spend weeks or months trying different products to determine for themselves which products are superior. Accordingly, there is a need for selling aids and techniques that can effectively and conveniently communicate product characteristics that translate into actual product performance when the product is used.

### SUMMARY OF THE INVENTION

According to one aspect of the present invention, an assortment or array of antiperspirant products is provided, of which at least some of the products are marketed as clinical strength, prescription strength, or the like. Typical medicaments can evolve from being available only by prescription to being available over-the-counter (“OTC”) without a prescription. The evolution can arise from positive usage and side effect data that builds over the life of the medicament. When a typical medicament makes the transition to OTC status and is marketed as “prescription strength,” consumers readily understand that the product will truly have a higher level of performance/efficacy as compared to a non-prescription strength alternative. Leveraging this understanding in atypical products, such as antiperspirants, can aid the consumer in choosing a better performing underarm product among the overwhelming number of options that are available on a store shelf.

In accordance with one of the preferred embodiments, there has now been provided an array of antiperspirant products, comprising a first antiperspirant product that is marketed to male consumers and marketed as clinical strength or prescription strength, and a second antiperspirant product that is marketed to female consumers and marketed as clinical strength or prescription strength.

In accordance with another preferred embodiment, there has now been provided an array of antiperspirant products, comprising a first antiperspirant product comprising a first brand name, the first antiperspirant product being marketed to male consumers and being marketed as clinical strength or prescription strength; a second antiperspirant product comprising a second brand name, the second antiperspirant product being marketed to female consumers and being marketed as clinical strength or prescription strength; a third antiperspirant product comprising the first brand name, the third antiperspirant product being marketed to male consumers and not being marketed as clinical strength or prescription strength; and a fourth antiperspirant product comprising the second brand name, the fourth antiperspirant product being marketed to female consumers and not being marketed as clinical strength or prescription strength.

In accordance with yet another preferred embodiment, there has now been provided an array of antiperspirant products, comprising a first antiperspirant product that is marketed to male consumers and includes directions to apply the product before sleeping, at or around nighttime, or at or around bedtime; and a second antiperspirant product that is marketed to female consumers and includes directions to apply the product before sleeping, at or around nighttime, or at or around bedtime.

### DETAILED DESCRIPTION OF THE INVENTION

While the specification concludes with the claims particularly pointing and distinctly claiming the invention, it is believed that the present invention will be better understood from the following description.

As used herein, “comprising” means that other steps and/or ingredients can be added. This term encompasses the terms “consisting of” and “consisting essentially of”. The phrase “consisting essentially of” means that the invention may include additional steps and/or ingredients, but only if the additional ingredients and/or steps do not materially alter the basic and novel characteristics of the claimed invention or methods.

All percentages, parts and ratios are based upon the total weight of the antiperspirant compositions of the present invention and all measurements made are at 25° C., unless otherwise specified. All such weights as they pertain to listed ingredients are based on the active level and, therefore; do not include carriers or by-products that may be included in commercially available materials, unless otherwise specified.

The term “ambient conditions,” as used herein, refers to surrounding conditions at about one atmosphere of pressure, 50% relative humidity and about 25° C.

The term “anhydrous” as used herein means that the antiperspirant compositions are substantially free of added or free water. For example, the antiperspirant compositions may comprise less than about 2%, less than about 1%, less than about 0.5%, or zero percent of free or added water, by weight of the composition.

Embodiments of the present invention involve a line or array of antiperspirant products having varying product performance characteristics and/or marketing targets. The product performance characteristic and/or marketing targets may include, but is not limited to, the product form, benefit of the product, antiperspirant efficacy (including actual and perceived wetness protection), the level of odor protection, fragrance levels, hardness, residue grade, and/or gender target. The product line may comprise at least a first product and a second product wherein the two products are differentiated and identified by an “identifier” selected from the group consisting of packaging indicia (words, graphics, or other markings), color of packaging, shelf marking and mixtures thereof. Unless stated otherwise, “packaging” includes primary packaging, secondary packaging, and mixtures thereof.

In at least some of the preferred embodiments, the array of antiperspirant products comprises first and second products that are designed to provide clinical or prescription strength wetness protection, with the first and second products being marketed to male and females respectively. As used herein, the terms “prescription strength” and “clinical strength” generally refer to an antiperspirant product that may have a significantly higher level of clinically measurable wetness protection, when used as directed, than is available in the same product form of a non-prescription strength or regular strength formulation. The terms “prescription strength” and “clinical strength” generally includes other similar labels such as “Dr. recommended,” “extra-strength,” “pharmacy grade,” and the like. The method for measuring clinical wetness protection is described in U.S. Pat. No. 6,352,688, issued to Scavone et al. on Mar. 5, 2002. In accordance with this method, antiperspirant products provided herein and that are marketed as “prescription strength” or “clinical strength” may exhibit a 3-day antiperspirant efficacy greater than about 1.35 and/or a 10-day antiperspirant efficacy that is greater than about 1.3. Of course, one of ordinary skill in the art will also be able to determine that if the efficacy level of a first product is greater than the efficacy level of a second product, the first product with the greater efficacy level may be considered “prescription strength” or “clinical strength” as compared to the second product.

It has been discovered that application of antiperspirant products prior to sleeping can improve their wetness protection. Sleeping may include rest for a period of time of at least about 1 hour, at least about 2 hours, at least about 4 hours or at least about 8 hours. Typical antiperspirant products work by forming temporary “plugs” in sweat ducts present in the skin, which can, in turn, reduce the flow of perspiration to the exterior of the skin. Once an antiperspirant is applied to the skin, perspiration in the underarm dissolves antiperspirant active particles, allowing the active to flow into the sweat ducts and form superficial plugs that are just below the surface of the skin. The body's core temperature typically varies throughout a 24-hour period, rising during the day and cooling during sleeping or resting. The rate at which humans sweat is affected by these temperature variations. Rapid and/or high levels of perspiration prior to forming effective plugs can decrease the level of antiperspirant active that is available for forming plugs by transferring the active away from the skin. Thus, application of antiperspirants prior to sleeping or resting facilitates the best probability of forming effective sweat duct plugs that can lead to wetness protection levels that may be characterized as “prescription strength” or “clinical strength.” In view of the foregoing, antiperspirant products and arrays of the same that are provided by the present invention may include directions to apply the product, for example, before sleeping, at or around nighttime, or at or around bedtime. Other language or directions that effectively communicate this application technique are considered to be within the scope of the present invention. For example, indicia that may be included on the higher efficacy level antiperspirants in the form of a label that indicates via words and/or graphics “Night.”

The array of antiperspirant products may contain both prescription/clinical strength products and regular or non-prescription/clinical strength products. The regular or non-prescription/clinical strength products may, for example, exhibit a may exhibit a 3-day antiperspirant efficacy that is less than about 1.35 and/or a 10-day antiperspirant efficacy that is less than about 1.3, according to the method for measuring clinical wetness protection described in U.S. Pat. No. 6,352,688. The distinction of wetness efficacy level may be communicated by text or other indicia that is included on the packaging and/or on product inserts or brochures. The distinction may alternatively or additionally be communicated by graphics, package design, color, or other visibly distinguishable technique.

The various antiperspirant products included in the array embodiments provided by the present invention may share a brand name. The various products may also be manufactured by, manufactured for, and/or distributed for the same company or company affiliate.

Antiperspirant products included within the arrays of the present invention generally include a liquid carrier material, an antiperspirant active, and one or more additional ingredients, such as, for example, thickening agents/gellants, perfumes (neat and/or encapsulated or complexed) that can attribute to product form, scent, and other product characteristics.

Suitable liquid carrier materials can include, but are not limited to, any topically safe and effective organic, silicone-containing or fluorine-containing, volatile or non-volatile, polar or non-polar liquid carrier. The liquid carrier is preferably liquid under ambient conditions, and can include one or more liquid carrier materials provided that the any such combination of materials is in liquid form under ambient conditions. Depending on the type of product form desired, concentrations of the liquid carrier in the compositions will typically range from about 10% or from about 30% to about 90% or to about 75%, by weight of the composition.

Nonlimiting examples of suitable liquid carriers include C₁ to C₂₀ monohydric alcohols, i.e., C₂ to C₈ monohydric alcohols; C₂ to C₄₀ dihydric or polyhydric alcohols, i.e., C₂ to C₂₀ dihydric or polyhydric alcohols; alkyl ethers of all such alcohols, i.e. C₁-C₄ alkyl ethers; and polyalkoxylated glycols, i.e. propylene glycols and polyethylene glycols having from 2 to 30 repeating alkoxylate (e.g., ethoxylate or propoxylate) groups and polyglycerols having from 2 to 16 repeating glycerol moieties; their derivatives and mixtures thereof.

Specific examples of such alcohol liquid carriers include propylene glycol, hexylene glycol, dipropylene glycol, tripropylene glycol, glycerin, propylene glycol methyl ether, dipropylene glycol methyl ether, ethanol, n-propanol, n-butanol, t-butanol, 2-methoxyethanol, 2-ethoxyethanol, ethylene glycol, isopropanol, isobutanol, 1,4-butylene glycol, 2,3-butylene glycol, trimethylene glycol, 1,3-butanediol, 1,4,-butanediol, propylene glycol monoisostearate, PPG-3 myristyl ether, PEG-4 (also known as PEG-200), PEG-8 (also known as PEG-400), 1,2, pentanediol, PPG-14 butylether, dimethyl isosorbide, and combinations thereof. Other similar but suitable solvents for use as liquid carriers are described, for example, in U.S. Pat. No. 4,781,917, issued to Luebbe et al., Nov. 1, 1998, U.S. Pat. No. 5,643,558, issued to Provancal et al., Jul. 1, 1997, U.S. Pat. No. 4,816,261, issued to Luebbe et al., Mar. 28, 1989 and EP 404 533 A1, published Dec. 27, 1990 by Smith et al.

The antiperspirant products may comprise a silicone liquid carrier. The concentration of the silicone liquid carrier may range from about 10% or from about 15% of a silicone liquid carrier, by weight of the composition to about 90% or to about 65% of a silicone liquid carrier, by weight of the composition. The silicone liquid carriers suitable for use herein may include volatile or non-volatile silicones.

Nonlimiting examples of suitable silicone liquid carriers for use herein include those volatile silicones that are described in Todd et al., “Volatile Silicone Fluids for Cosmetics”, Cosmetics and Toiletries, 91:27-32 (1976). Suitable amongst these volatile silicones include the cyclic silicones having from about 3 or from about 4 to about 7 or to about 6, silicon atoms. Specifically are those which conform to the formula: 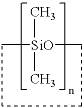
wherein n is from about 3, from about 4 or about 5 to about 7 or to about 6. These volatile cyclic silicones generally have a viscosity value of less than about 10 centistokes.

Other suitable silicone liquid carriers for use herein include those volatile and nonvolatile linear silicones which conform to the formula: 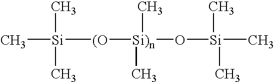
wherein n is greater than or equal to 0. The volatile linear silicone materials will generally have viscosity values of less than 5 centistokes at 25° C. The non-volatile linear silicone materials will generally have viscosity values of greater than 5 centistokes at 25° C.

Specific examples of suitable volatile silicones for use herein include, but are not limited to, hexamethyldisiloxane; Silicone Fluids SF-1202 and SF-1173 (commercially available from G.E. Silicones); Dow Corning 244, Dow Corning 245, Dow Corning 246, Dow Corning 344, and Dow Corning 345, (commercially available from Dow Corning Corp.); Silicone Fluids SWS-03314, SWS-03400, F-222, F-223, F-250, and F-251 (commercially available from SWS Silicones Corp.); Volatile Silicones 7158, 7207, 7349 (available from Union Carbide); Masil SF-V™ (available from Mazer); and mixtures thereof.

Specific examples of suitable non-volatile linear silicones for use herein include, but are not limited to, Rhodorsil Oils 70047 available from Rhone-Poulenc; Masil SF Fluid available from Mazer; Dow Corning 200 and Dow Corning 225 (available from Dow Corning Corp.); Silicone Fluid SF-96 (available from G.E. Silicones); Velvasil™ and Viscasil™ (available from General Electric Co.); Silicone L-45, Silicone L-530, and Silicone L-531 (available from Union Carbide); and Siloxane F-221 and Silicone Fluid SWS-101 (available from SWS Silicones).

Other suitable non-volatile silicone liquid carriers for use in the antiperspirant products of the present invention include, but are not limited to, non-volatile silicone emollients such as polyalkylarylsiloxanes, polyestersiloxanes, polyethersiloxane copolymers, polyfluorosiloxanes, polyaminosiloxanes, and combinations thereof. These non-volatile silicone liquid carriers will generally have viscosity values of less than about 100,000 centistokes, less than about 500 centistokes, or from about 1 centistokes to about 200 centistokes or to about 50 centistokes, as measured under ambient conditions.

Other suitable liquid carriers for use in the antiperspirant products include, but are not limited to, organic liquid carriers such as mineral oil, petrolatum, isohexadecane, isododecane, various other hydrocarbon oils, and mixtures thereof. Preferred are mineral oil and branched chain hydrocarbons having from about 4 or from about 6 carbon atoms to about 30 or to about 20 carbon atoms. Specific non-limiting examples of suitable branched chain hydrocarbon oils include isoparaffins available from Exxon Chemical Company as Isopar C™ (C7-C8 Isoparaffin), Isopar E™ (C8-C9 Isoparaffin), Isopar G™ (C10-11 Isoparaffin), Isopar H™ (C11-C12 Isoparaffin), Isopar L™ (C11-C13 Isoparaffin), Isopar M™ (C13-C14 Isoparaffin), and combinations thereof. Other nonlimiting examples of suitable branched chain hydrocarbons include Permethyl™ 99A (isododecane), Permethyl™ 102A (isoeicosane), Permethyl™ 101A (isohexadecane), and combinations thereof. The Permethyl™ series are available from Preperse, Inc., South Plainfield, N.J., U.S.A. Other non-limiting examples of suitable branched chain hydrocarbons include petroleum distillates such as those available from Phillips Chemical as Soltrol™ 130, Soltrol™ 170, and those available from Shell as Shell Sol™ 70, -71, and -2033, and mixtures thereof.

Examples of other suitable organic liquid carriers include the Norpar™ series of paraffins available from Exxon Chemical Company as Norpar™ 12, -13, and -15; octyldodecanol; butyl stearate; diisopropyl adipate; dodecane; octane; decane; C₁-C₁₅ alkanes/cycloalkanes available from Exxon as Exxsol™ D80; C₁₂-C₁₅ alkyl benzoates available as Finsolv-TN™ from Finetex; and mixtures thereof. Other suitable liquid carriers include benzoate co-solvents, cinnamate esters, secondary alcohols, benzyl acetate, phenyl alkane, and combinations thereof.

The antiperspirant products of the present invention may be formulated as an aqueous or anhydrous composition. Aqueous compositions may comprise from about 10% or from about 15% water, by weight of the composition to about 75%, to about 60%, or to about 50% water, by weight of the composition. Anhydrous compositions may comprise less than about 10%, less than about 3%, less than about 1%, or zero percent water, by weight of the composition.

The antiperspirant active for use in the antiperspirant products may include any compound, composition or other material having antiperspirant activity. By way of example only, the antiperspirant actives may include astringent metallic salts, especially inorganic and organic salts of aluminum, zirconium and zinc, as well as mixtures thereof. Particular antiperspirant active examples include, but are not limited to, aluminum-containing and/or zirconium-containing salts or materials, such as aluminum halides, aluminum chlorohydrate, aluminum hydroxyhalides, zirconyl oxyhalides, zirconyl hydroxyhalides, and mixtures thereof.

Aluminum salts useful in the present invention include those that conform to the formula:
Al₂(OH)ₐCl_{b}.x H₂O
wherein a is from about 0 to about 5; the sum of a and b is about 6; x is from about 1 to about 8; where a, b, and x may have non-integer values. For example, aluminum chlorohydroxides referred to as “¾ basic chlorohydroxide,” wherein a is about 4.5; “⅚ basic chlorohydroxide”, wherein a=5; and “⅔ basic chlorohydroxide”, wherein a=4 may be used. Processes for preparing aluminum salts are disclosed in U.S. Pat. No. 3,887,692, issued to Gilman on Jun. 3, 1975; U.S. Pat. No. 3,904,741, issued to Jones et al. on Sep. 9, 1975; and U.S. Pat. No. 4,359,456 issued to Gosling et al. on Nov. 16, 1982. A general description of these aluminum salts can also be found in Antiperspirants and Deodorants, Cosmetic Science and Technology Series Vol. 20, 2nd edition, edited by Karl Laden. Mixtures of aluminum salts are described in British Patent Specification 1,347,950, filed in the name of Shin et al. and published Feb. 24, 1974.

Zirconium salts for use in the present invention include those which conform to the formula:
ZrO(OH)₂₋ₐClₐ.x H₂O
wherein a is from about 0.5 to about 2; x is from about 1 to about 7; where a and x may both have non-integer values. These zirconium salts are described in Belgian Patent No. 825,146, issued to Schmitz on Aug. 4, 1975. Useful to the present invention are zirconium salt complexes that additionally contain aluminum and glycine, commonly known as “ZAG complexes”. These complexes contain aluminum chlorohydroxide and zirconyl hydroxy chloride conforming to the above-described formulas. Such ZAG complexes are described in U.S. Pat. No. 4,331,609, issued to Orr on May 25, 1982 and U.S. Pat. No. 4,120,948, issued to Shelton on Oct. 17, 1978.

In one preferred embodiment, the “prescription strength” or “clinical strength” product within the array of antiperspirant products is substantially devoid of aluminum chloride as the antiperspirant active. In alternative embodiments, the antiperspirant products may contain aluminum chloride.

Antiperspirant products of the present invention may optionally contain one or more fragrance materials. Scent expression or release technology may be employed with some or all of the fragrance materials to define a desired scent expression prior to use and during use of the antiperspirant products. Such scent expression or release technology can include cyclodextrin complexing material. Other materials, such as, for example, starch-based matrices may be employed to “hold” fragrance materials prior to exposure to bodily-secretions (e.g., perspiration). The encapsulating material may have release mechanisms other than via a solvent; for example, the encapsulating material may be frangible, and as such, rupture or fracture with applied shear and/or normal forces encountered during application and while wearing.

The antiperspirant products may optionally comprise thickening agents to help provide the compositions with the desired viscosity, rheology, texture and/or product hardness, or to otherwise help suspend any dispersed solids or liquids within the composition. The term “thickening agent” may include any material known or otherwise effective in providing suspending, gelling, viscosifying, solidifying or thickening properties to the composition or which otherwise provide structure to the final product form. These thickening agents may include gelling agents, polymeric or nonpolymeric agents, inorganic thickening agents, or viscosifying agents. The thickening agents may include organic solids, silicone solids, crystalline or other gellants, inorganic particulates such as clays or silicas, or combinations thereof.

The antiperspirant products may further comprise one or more optional components which may modify the physical or chemical characteristics of the compositions or serve as additional “active” components when deposited on the skin. Nonlimiting examples of such optional materials include, but are not limited to, pH buffering agents, additional malodor controlling agents such as deodorant actives, fragrance materials, emollients (e.g., glycerin), antioxidants, humectants, soothing agents, dyes and pigments, medicaments, baking soda and related materials, preservatives, and soothing agents such as aloe vera, allantoin, D-panthenol, pantothenic acid derivatives (e.g., those disclosed in U.S. Pat. No. 6,495,149), avocado oil and other vegetative oils, and lichen extract.

The skilled artisan can make the antiperspirant products by any known or otherwise effective technique. Many such techniques are described in the antiperspirant/deodorant formulation arts.

The array of antiperspirant products of the present invention may be packaged individually in separate containers or they may be packaged together in a unitary form to be sold and bought together. Written and/or graphic instructions may be included in the separate packages or in the unitary packages to instruct a consumer when and how to use the product. Additionally, the product or products may be packaged in a secondary package wherein an outer container embodies the product disclosed therein.

The antiperspirant products may be applied topically to the underarm or other suitable area of the skin in an amount effective to reduce or inhibit perspiration wetness. The products are preferably applied in an amount ranging from at least about 0.1 gram but no more than about 20 grams, no more than about 10 grams, or no more than about 1 gram.

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “40 mm” is intended to mean “about 40 mm”.

All documents cited in the Detailed Description of the Invention are, are, in relevant part, incorporated herein by reference; the citation of any document is not to be construed as an admission that it is prior art with respect to the present invention. To the extent that any meaning or definition of a term in this document conflicts with any meaning or definition of the term in a document incorporated herein by reference, the meaning or definition assigned to the term in this document shall govern.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

What is claimed is:

1. An array of antiperspirant products, comprising:
a first antiperspirant product that is marketed to male consumers and marketed as clinical strength or prescription strength; and
a second antiperspirant product that is marketed to female consumers and marketed as clinical strength or prescription strength,
wherein the first antiperspirant product and the second antiperspirant product share a brand name,
wherein each of the first antiperspirant product and the second antiperspirant product comprises an antiperspirant active that is substantially devoid of aluminum chloride,
wherein each of the first antiperspirant product and the second antiperspirant product comprises fragrance materials, and
wherein each of the first antiperspirant product and the second antiperspirant product comprises both primary and secondary packaging.

2. The array of antiperspirant products according to claim 1, wherein fragrance release technology is employed with some or all of the fragrance materials.

3. The array of antiperspirant products according to claim 2, wherein the fragrance release technology comprises starch.

4. The array of antiperspirant products according to claim 1, further comprising a third antiperspirant product that is not marketed as clinical strength or prescription strength, the third antiperspirant product sharing a brand name with the first antiperspirant product and the second antiperspirant product.

5. The array of antiperspirant products according to claim 1, wherein each of the first antiperspirant product and the second antiperspirant product comprises instructions to apply the product at or around bedtime so that a consumer can understand how to achieve the clinical or prescription strength protection.

6. An array of antiperspirant products, comprising:
a first antiperspirant product comprising a first brand name, the first antiperspirant product being marketed to male consumers and being marketed as clinical strength or prescription strength;
a second antiperspirant product comprising a second brand name, the second antiperspirant product being marketed to female consumers and being marketed as clinical strength or prescription strength;
a third antiperspirant product comprising the first brand name, the third antiperspirant product being marketed to male consumers and not being marketed as clinical strength or prescription strength; and
a fourth antiperspirant product comprising the second brand name, the fourth antiperspirant product being marketed to female consumers and not being marketed as clinical strength or prescription strength,
wherein each of the first antiperspirant product and the second antiperspirant product comprises fragrance materials, and
wherein each of the first antiperspirant product and the second antiperspirant product comprises both primary and secondary packaging.

7. The array of antiperspirant products according to claim 6, wherein fragrance release technology is employed with some or all of the fragrance materials.

8. The array of antiperspirant products according to claim 7, wherein the fragrance release technology comprises starch.

9. The array of antiperspirant products according to claim 6, wherein each of the first antiperspirant product and the second antiperspirant product comprises instructions to apply the product at or around bedtime so that a consumer can understand how to achieve the clinical or prescription strength protection.

10. The array of antiperspirant products according to claim 6, wherein each of the first antiperspirant product and the second antiperspirant product comprises an antiperspirant active that is substantially devoid of aluminum chloride.
